(19) United States
(12) Patent Application Publication
(10) Pub. No.: US 2005/0266064 A1
(43) Pub. Date: Dec. 1, 2005

(54) Cosmetic compositions and methods

(76) Inventor: Kathryn Jeanette McCarthy, Valencia, CA (US)

Correspondence Address: KATHY J. McCARTHY, 26810 PINE CLIFF PL., VALENCIA, CA, 91381, US

(21) Appl. No.: 10/857,766

(22) Filed: May 29, 2004

(51) Int. Cl. A61K 31/198; A61K 31/401; A61K 31/522; A61K 35/78; A61K 9/127

(52) U.S. Cl. 424/450; 424/739; 424/760; 514/263.31; 514/423; 514/564; 514/474

(57) ABSTRACT

Methods and compositions are provided for enhancing the size of human lips as well as for reducing fine lines and wrinkles in the skin with topical applications. Methods and compositions are disclosed for increasing blood flow and stimulating collagen synthesis in the skin and surrounding tissues. Methods and compositions are disclosed for increasing storage and release of lip size enhancing ingredients with extended release delivery systems to overcome the reduced layers of skin on the lips. Methods and topical compositions are provided for increasing the rate of new tissue formation in the lips. Methods and topical compositions are disclosed for improving the appearance, firmness and vitality of skin and surrounding tissues.

### BACKGROUND-FIELD OF INVENTION

The invention relates to compositions and methods for enhancing lips, specifically to topical compositions and methods used for enhancing lip appearance in people. The invention also relates to topical compositions and methods used for reducing the effects of aging and reducing the appearance of wrinkles.

### BACKGROUND-DESCRIPTION OF PRIOR ART

Many prior art methods for lip enhancement include surgical approaches such as implants or injections of collagens, silicones, fats, hyaluronic acid derivatives, bovine compositions or other injected material. Surgical implants or injections can create risk of infection, internal tissue damage, scar tissue, discoloration, internal negative reactions, damage to the dermal layers, post surgical pain and extended recovery.

Prior art topical compositions and methods for lip enhancement do not provide significant increase in the size and fullness of lips. This often causes people to switch from brand to brand only to find out that none of these topical compositions truly increase the size and fullness of their lips. This often causes people to consider surgical alternatives such as lip injections to achieve the level of fullness they desire.

Many prior art topical compositions for lip enhancement list in the product ingredients the use of cinnamon or cinnamon oil to irritate the mucosa and to cause dilation of the capillaries; however, the small amount of swelling goes away after a short period, often after a few minutes.

While some prior art topical compositions for lip enhancement use ingredients designed to stimulate collagen and hyaluronic acid formation, these compositions often take 30 or more days of repetitive treatments to begin seeing some results.

Prior art topical compositions for lip enhancements do not adequately address the difficulties of increasing lip size created by the highly elastic nature of the lip tissues which can cause the lip tissues to quickly return to their normal size after a short period of temporary swelling induced by a topical composition; and therefore, both the short term and long term effectiveness of prior art lip volume enhancement compositions are significantly inhibited.

Another disadvantage of prior art topical lip volume enhancement compositions is that they do not adequately address the problem that the skin of the lips has fewer layers than the skin on the rest of the body and lacks the protective layer called cornified tissue. Therefore, the skin of the lips has reduced ability for holding and retaining topical compounds. Furthermore, beneath this thin skin of the lips are numerous blood vessels (which make the lip look red) and these blood vessels can carry lip enhancing compounds away from the lip tissues so that effectiveness is reduced and increases in volume can be short lived.

The disadvantages and low effectiveness of many prior art topical lip volume enhancement compositions often cause consumers to give up hope after trying numerous versions and choose surgical injections or implants which can create serious risks and dangers.

### OBJECTS AND ADVANTAGES

Accordingly, in addition to the objects and advantages of lip enhancing compositions and methods described in this specification, several objects and advantages of the methods and compositions of the present invention can include:
(a) to provide topical lip volume enhancement methods and compositions that offer increased effectiveness;
(b) to provide methods for extended release and automatic replenishment of lip volume enhancing compounds over extended durations so as to significantly reduce the need for reapplication;
(c) to provide topical lip volume enhancement methods and compositions that provide significantly long durations of lip volume increase;
(d) to provide methods for increasing the duration of lip tissue swelling in an amount effective to permit the lip tissues to expand and adjust to the increased volume so as to reduce the amount of elastic recovery after swelling and to permit lip tissues to experience increased net gain in volume over time;
(e) to provide methods for storing and releasing lip volume enhancing compounds and lip tissue nourishing compounds over extended periods with extended release delivery systems so as to substantially overcome the low storage capacity of compounds on and within the thin skin layers of the lips;
(f) to provide methods for storing and releasing lip volume enhancing compounds and lip tissue nourishing compounds over extended periods with extended release delivery systems so as to substantially permit the automatic extended replenishment of lip volume enhancing compounds and lip tissue nourishing compounds to the blood vessels and tissues beneath the thin skin of the lip as the vast network of blood vessels beneath the thin skin of the lips begins to absorb and carry such lip volume enhancing compounds and lip tissue nourishing compounds away from the lip tissues;
(g) to provide methods improving the effectiveness of topically applied blood circulation enhancing compounds that normally taper off in effectiveness after a relatively short period of time, in which additional amounts of such compounds are stored within extended release delivery systems for automatic re-application on a time release basis substantially coinciding with the time that the original unstored compounds begin to taper off in effectiveness, or coinciding with the time that previously released stored circulation enhancing compounds begin to taper off in effectiveness, thereby enabling such short period blood circulation enhancing compounds to be effective for significantly increased periods capable of creating longer durations of increased blood flow, lip volume enhancement and lip tissue reorganization;
(h) to provide methods for extended release delivery of blood flow enhancing compounds for increasing the duration of increased circulation and swelling to the lip tissues in an amount effective to increase new blood vessel formation within the lip tissues;
(i) to provide methods for extended release delivery of topical collagen and hyaloronic acid production formation stimulating compounds in an amount effective to increase the formation of new collagen for increased tissue formation within the lips;
(j) to provide methods for extended release delivery of blood circulation enhancing compounds and topical collagen and hyaloronic acid production stimulation compounds to the lip in an amount effective to increase the formation of new collagen and hyaloronic acid within the lips during the time that the extended release of blood circulation enhancing compounds are creating an extended increase lip volume, thereby permitting such collagen and hyaloronic acid to begin forming while the lip tissues are in an expanded condition for greater lip enhancement effectiveness;
(k) to provide methods for extended release delivery of blood circulation enhancing compounds and collagen and hyaloronic acid formation stimulating compounds within a carrier containing hydration enhancing compounds for delivering simultaneous hydration to the lip tissues blood circulation stimulation and collagen formation stimulation while providing hydration enhancing compounds maximizing lip hydration;
(l) to provide methods and compounds that store and release capsicum with at least one extended release delivery system disposed within a topical carrier for application to the lips and arranged to provided an extended release of such capsicum in an amount and duration effective to significantly increase lip size and volume due to swelling and increase blood flow to the lip tissues;
(m) to provide methods and compounds that store and release methyl-sulfonyl-methane with at least one extended release delivery system disposed within a topical carrier for application to the lips and arranged to provide an extended release of such methyl-sulfonyl-methane in an amount and duration effective to increase elasticity of the lip tissues;
(n) to provide methods and compounds that store and release of methyl-sulfonyl-methane with at least one extended release delivery system disposed within a topical carrier for application to the lips and arranged to provide an extended release of such methyl-sulfonyl-methane in an amount and duration effective to stimulate collagen and hyaloronic acid formation within the lip tissues;
(o) to provide growth factors and, or growth hormones for increasing collagen synthesis and new tissue formation;
(o) to provide methods and topical compositions which offer improved ability to reduce the appearance of aging in the skin;
(p) to provide methods and topical compositions which offer improved reduction of wrinkles and fine lines in the skin;
(q) to provide methods and topical compositions which offer improved moisturizing benefits to the skin; and
(q) to provide methods and topical compositions capable of improving the formation of collagen and hyaluronic acid within and around the tissues of the skin.

Further objects and advantages will become apparent from a consideration of the ensuing description. While description contains various biological and chemical theories, the inventor intends that the present invention should not be limited or bound by such theories.

### DESCRIPTION

To achieve desired results, a preferred embodiment of the present invention uses the method of storing and releasing lip volume enhancing compounds and nutrients with a suitable extended release delivery system disposed within a topical carrier suitable for application to the lips. It is preferred that the extended release system provide sufficient storage and release to permit automatic reapplication of such compounds to substantially overcome the poor capacity for storage of compounds within the skin of the lips due to the extreme thinness and reduced layers of the skin of the lips. It is also preferred that such extended release delivery system would provide sufficient storage and release of such lip enhancing compounds to the lip tissues so as to substantially compensate for any tendency for the blood vessels within the lips to flush such compounds away from the lip area and reduce their concentration after initial application. It is also preferred that such extended release delivery systems provide sufficient storage and release of short period lip enhancing compounds to permit the strategic release of new amounts of such short period lip enhancing compounds after the initial application or subsequent releases have reached the end of their period of effectiveness so that such compounds are no longer limited by their original limited time period or cycle of effectiveness. It is preferred that extended release delivery systems provide a sufficient amount and duration for the release of such lip enhancing compounds so as to permit new tissue generation or augmentation to begin occurring during and, or after a period of increased blood flow. Any concentration of any ingredient sufficient to achieve this effect may be used.

Any suitable extended release delivery system may be used. For example, extended release delivery systems can include time activated release systems, heat activated release systems, pH activated release, solvent dependent release, moisture activated release systems, or any other suitable method of release. Extended release systems can include single delivery systems, multiple delivery systems and combinations of delivery systems. Some examples of possible extended release delivery systems can include capsules, micro capsules, crystals, conglomerates, gelatins, bubbles, powders, graduals, bubbles, micro bubbles, micro balloons, hydrogels and liposomes.

A preferred embodiment would use liposomes since the liposome wall is very similar, physiologically, to the material of human cell membranes. This can allow the liposomes containing the lip enhancing compounds to be deposited on the skin of the lip and begin to merge with the cellular membranes of the lip tissue while releasing the payload of lip enhancing compounds. The delivery can take place directly to the intended cells and can occur over a longer period of time. Liposomes as a delivery system can be made to release the lip volume enhancing compositions in a variety of ways including a slow as well as fast release of a hydrophilic payload (or any desired type of predetermined payload), a slow as well as fast release of a predetermined payload, bilayer composition (such as chain length, saturation, or lipid class), arranging the physical configuration of the liposomes for desired release, providing solvent dependent release, providing a pH dependent release as well providing a temperature dependent release. Liposomes may also be arranged to deliver predetermined payloads to various skin and tissue depths within and around the lips. Types of liposomes can include stabilized lecithin mixtures, synthetic identical-chain phospholipids, glycolipid containing liposomes, bipolar fatty acids, antibody directed, methyl/methylene x-linked, lipoprotein coated, carbohydrate coated, multiple encapsulated and emulsion compatible liposomes; however, any suitable form may be used. Preferably, the liposomes can be added to the desired formulation below 40° C. using low shear mixing; however, any suitable process may be used. It is also preferred that the addition of liposomes would be at or near the last step in the formulation's manufacturing process to maximize stability of the liposomes; however, any suitable step may be used.

Whatever the extended release system or systems that are used, a preferred embodiment would include at least one blood flow circulation stimulating compound to be stored and released from such extended release system. It is preferred that such an extended release system has a sufficient amount and duration of release of blood flow circulation stimulating compounds to stimulate angiogenesis or new blood vessel formation within the tissues of the lips. Such new blood vessel formation can create increased lip volume, or to reduce the appearance of fine lines and wrinkles, or to increase moisture and vitality to the skin. Such blood flow circulation stimulating compounds can also be used to stimulate collagen formation within the skin and surrounding tissues. A preferred circulation stimulant is a capsicum or capsacian. Capsicum or capsaicin can be in the form of cayenne pepper, cayenne extract, capsicum annuum, capsicum frutescens, or suitable extracts of any type of hot peppers such as jalapeno peppers, banana peppers, scotch bonnet peppers, habanero peppers or any other suitable hot pepper, either from the plant, the fruit or the seeds. The use of the words capsicum and capsaicin in this description are used to describe extracts of hot peppers and it is intended that the use of one of these words can also include the other word for the purposes of this description. Any concentration sufficient to increase blood circulation may be used. For example, concentrations by weight of capsicum or capsaicin may range between 0.01% to 1%, 1% to 3%, 3% to 5%, 5% to 10%, and higher than 10%. Preferably, the type of capsicums selected will occur at concentrations sufficiently high enough to increase blood circulation, yet sufficiently low enough to prevent intolerable pain. Preferably, such concentrations would be arranged to increase blood flow in an amount sufficient to create at least a temporary increase in the volume of the lip tissues and, or surrounding tissues. It is preferred that some amount of a tolerable, yet significant burning sensation and, or tingling sensation to occur in order to maximize a short term increase in lip volume; however, alternate preparations may use concentrations that produce minimal or even no noticeable burning or tingling sensation. In alternate embodiment compositions used for reducing the effects of aging or reducing the appearance of wrinkles in other areas of skin than that lips, concentration can be lower to provide reduced tingling or burning sensation or even no tingling or burning sensation at all, while preferably still having concentrations sufficient to stimulate some degree of increased blood flow and circulation.

Other preferred blood circulation stimulants that can be used in any combination with capsicum, in any combination with each other without including a capsicum, or as a single blood circulation stimulating compound without including a capsicum may include: caffeine, cinnamon, cinnamon leaf oils, cinnamon bark extracts or oils, menthol, camphor, niacin, coffee extracts, comfrey root, spearmint leaf/leaves, spearmint oil, ginger root, ginger, ginger extract, wasabi extracts, radish extract, guarana extract, ginseng extracts, mimosine (from the mimosa pudica plant), onion extracts, tea tree oil, sepitonic (copper, zinc, and magnesium which helps increase microcirculation aiding in optimal function of the skin), gatuline (beech bud, which aids in oxygenation of the skin), glycocides, stevia extracts, stevia leaf extracts, steviocides, auitable retinoids (may include retinol, retinoic acid, retinolic acid, Retin-A®, Retirides®, retinyl palmitate, reinyl propionate, retinyl acetate, isotrentonin, and the like), camphor, cajeput oil, dementholized mint oil, cassia oil, or any other suitable blood circulation stimulating compound or extract capable of increasing blood circulation.

As an example, sufficient amounts of stevia extracts, stevia leaf extracts, or steviocides can be used within a suitable topical carrier to increase blood circulation within the skin and, or surrounding tissues. In embodiments used for reducing wrinkles and fine lines, sufficient amounts of stevia extracts, stevia leaf extracts, or steviocides can be used within a suitable topical carrier to reduce the appearance of wrinkles and fine lines. In embodiments used for moisturizing skin, sufficient amounts of stevia extracts, stevia leaf extracts, or steviocides can be used within a suitable topical carrier to provide the skin with increased firmness, elasticity, vitality or moisture retention.

Any concentrations sufficient to increase blood circulation may be used. When using combinations of blood circulation stimulating ingredients or compounds, it is preferred that the concentrations of the total combined blood circulation stimulating ingredients are sufficient to increase blood circulation and individual ingredients may be included at any concentration. In alternate embodiments, each individual blood circulation stimulating ingredient may be at a sufficient concentration to stimulate blood flow on its own, regardless of the presence or lack of presence of other blood circulation stimulating ingredients.

To achieve preferred results, a preferred embodiment would exhibit blood flow circulation stimulant properties over extended periods of time by having blood flow circulation stimulating compounds arranged to be stored and released by at least one extended release delivery system disposed within a topical carrier suitable for application to the lips; or in alternate embodiments, for application to the skin for reducing fine lines and wrinkles. For lip enhancing embodiments, extended release to the mucosa, lip or transdermal lip area can augment the lip by circulating the blood flow of the lip for a significantly longer duration of time without having to reapply the composition in short intervals so that the lips can stay fuller longer. Furthermore, the increased duration permits tissues to adjust and new blood vessels to begin forming while during an extended state of swelling so as to permit long term gain in lip size and tissue to accumulate with increased efficiency and improved results. Preferably, the concentration and delivery should be sufficient to stimulate blood circulation within the lip tissues and mucosa so as to create a noticeable increase in lip size and the extended release system should allow the duration of such increase in lip size to be significantly extended. It is preferred that an extended release delivery system releases lip volume enhancing compounds for a period of at least 30 minutes after application. It is preferred that longer durations of release are achieved for greater effectiveness. Examples of such longer durations can be at least 45 minutes after application, at least 60 minutes after application, or at least 90 minutes after applications. Preferably, periods of effective extended released will be greater than one hour after application and will ideally continue for several hours after application. Preferably, the duration of swelling should be sustained for approximately 45 minutes, approximately one hour, for several hours or even many hours. Single or multiple extended release phases can be arranged in the compositions. In one preferred embodiment, lip enhancing compositions can be released upon application with further amounts being released in various predetermined intervals. Predetermined intervals can include intervals of 5 minutes, 15 minutes, 20 minutes, 30 minutes, 40 minutes, 45 minutes and greater than 50 minutes, and numerous repetitions of such intervals can be arranged. Repetitive intervals can be established by having varied phases of release. For example, to have releases at intervals of 15 minutes, then a predetermined amount is released immediately upon application, additional amounts are released 15 minutes after application, 30 minutes after application, 45 minutes after application, 60 minutes after application, 75 minutes after application and so on. Another example, to have releases at intervals of 30 minutes, then a predetermined amount is released immediately upon application, additional amounts are released 30 minutes after application, 60 minutes after application, 90 minutes after application and so on. The intervals can be either consistent or varied according to desire. Some lip enhancing ingredients may be released during the same release phase as other ingredients or at different phases or intervals than other ingredients in any manner desired. Other lip enhancing compounds may be released in addition to or in lieu of blood circulation enhancing ingredients either in single or multiple extended release phases. Any suitable lip volume enhancing ingredient or nutritive ingredient can be included in general solution or suspension in the composition, and can also be supplied in extended release deliver systems. In lip volume enhancing embodiments, these ingredients and delivery systems may be used with concentrations sufficient to create an increase in the volume of the lips.

In alternate embodiment compositions used for reducing fine lines and wrinkles in other areas of the skin besides the lips, the time release delivery systems can be used to store at least one release blood flow stimulating ingredient, at least one collagen synthesis stimulating ingredient, at least one hyaluronic acid formation stimulating ingredient, at least one elastin formation stimulating ingredient, or at least one glucosamnoglycan formation stimulating ingredient, as well as any combination of such ingredients. Concentrations of such ingredients would preferably be sufficient to stimulate angiogenesis or new blood vessel formation within the tissues in and around fine lines and wrinkles in an amount effective to reduce the appearance of wrinkles and fine lines over repeated applications.

Preferred embodiments would preferably include compounds existing within the composition in general solution, suspension and, or extended release delivery systems for stimulating the formation of collagen, hyaluronic acid, elastin and glucosamnoglycans within the skin and surrounding tissues with concentrations sufficient to permit lip volume to become increased or to permit wrinkles and, or fine lines to be reduced. Examples of collagen enhancing ingredients may include collagen, water soluble collagen, marine collagen or other sources of collagen, elastin, marine elastin or other sourches of elastin, hyaluronic acid, collagen precursors, procollagens (such as procollagen type I for example), hydroxproline, palmitoyl oligopeptide, hyaluronic acid salts (such as sodium salts), any suitable form of hyaluronic acid and derivatives or precursors thereof. These and other compounds can stimulate collagen formation in the tissues on and surrounding the areas of the lip to increase lip volume and, or to reduce fine lines and wrinkles in other areas of the skin. Any ingredient or ingredients suitable for stimulating the formation of collagen, hyaluronic acid, elastin, and glucosamnoglycans within the skin or tissues may be used in addition to or in substitution to any of these ingredients. For example, such other ingredients may include hylaform (a slightly different form of hyaluronic acid derived from rooster combs), allantoin, niacin, methyl-sulfonyl-methane (MSM) and suitable derivatives thereof. Preferably, methyl-sulfonyl-methane (MSM) and/or any other suitable collagen stimulating ingredient can be delivered by a suitable extended release deliver system for extended release with predetermined release intervals to permit extended storage and continued replenishment to the lips for enhanced collagen stimulation and new tissue formation. Preferred embodiments of the present invention uses methyl-sulfonyl-methane (MSM) to enhance lip volume, support collagen synthesis and support new tissue growth. For example, liposomes could be used to deliver methyl-sulfonyl-methane (MSM) to the target cells over extended durations for enhanced effectiveness.

Other collagen stimulating ingredients can also include extracts or secretions from snails, shell fish, mollusks, plankton, and sea vegetables. For example, extracts or secretions from the snail helix aspersa miller contain collagen, elastin, allantoin, vitamins, keratin, glycolic acid, natural antibiotics and other compounds. As another example, sufficient amounts of stevia extracts, stevia leaf extracts, or steviocides can be used within a suitable topical carrier to stimulate new tissue formation and collagen synthesis. In embodiments used for reducing wrinkles and fine lines, sufficient amounts of stevia extracts, stevia leaf extracts, or steviocides can be used within a suitable topical carrier to reduce the appearance of wrinkles and fine lines. In embodiments used for moisturizing skin, sufficient amounts of stevia extracts, stevia leaf extracts, or steviocides can be used within a suitable topical carrier to provide the skin with increased firmness, elasticity, vitality or moisture retention.

Further collagen stimulating compounds can include vitamins such as vitamin C, amino acids, and minerals. Preferred embodiments would include vitamin C, preferably in an extended release delivery system such as liposomes. Any form of vitamin C may be used including vitamin C esters. In a preferred embodiment, vitamin C is protected within the composition from destructive environmental conditions such as oxidizing agents with a predetermined environmental protective element, component or system so that preferably the vitamin C does not oxidize and remains effective in promoting collagen synthesis and countering free radicals. Preferably, suitable extended release delivery systems, such as liposomes are arranged to protect the vitamin C (as well as other compounds) from environmental destruction prior to entering the cell tissues. Preferably, vitamin C concentrations are preserved by sufficient amounts of protective substances and are arranged to be quickly absorbed by the skin and lip tissues upon release from the extended release delivery system so that the vitamin C does not deteriorate and can effect the cell while intact, and are sufficiently high enough in concentration to increase vitamin C levels in the lip tissue. Preferably, concentrations of vitamin C are sufficient to increase collagen synthesis in an amount effective to increase lip volume over time.

Numerous other collagen stimulating compounds may be included individually or in combinations. Various bioflavinoids and antioxidants may be used as well. Bioflavinoids can be used as a bridging agent to help deliver vitamin C to the skin cells with increased effectiveness and activity. Magnesium ascorbyl phosphate can be used to help stimulate collagen and help repair moisture zones. Ascorbyl methylsilanol pectinate has stability from silicon and can be used as well. Other antioxidant support can include grape seed extract, citris flavinoids and bioflavinoids, vitamine E, beta carotene, vitamin A, green tea extract, pomegranate extracts, bilberry extract, cranberry extract, blueberry extract, raspberry extract, berry extracts, fruit extracts, vegetable extracts, pine bark extracts, pycnogenol® (French maritime pine bark extract), coenzyme Q-10, wheat protein, alpha-lipoic acid, N-acetylcysteine, selenium, rosemary extract, lycopene, and glutathione.

In one preferred embodiment using vitamin E, both vitamin C and alpha-lipoic acid can be used in the lip composition to help change radical forms of vitamin E back to its antioxidant form. Should the vitamin C change to a free radical when converting the vitamin E to an antioxidant form, glutathione can be used in this combination as well so that the alpha-lipoic acid and glutathione in the lip enhancing composition can help convert vitamin C back into the desired antioxidant form for increased collagen formulation. N-acetylcysteine can be supplied in the lip enhancing composition to help generate glutathione, and alpha-lipoic acid can be used to help re-create glutathione for increased collagen formation. Selenium can be used in the lip enhancing compound in any suitable form such as the enzyme glutathione peroxidase to help recycle glutathione. Other vitamins can be used as well, including vitamin K for blood vessel formation support. Other suitable vitamins can include vitamin B6, vitamin B12, Vitamin D-3, 1,25-dihydroxy vitamin D3, vitamin B1, vitamin B2, vitamine F, tocotrienols and their derivatives, nicotinic acid and its esters, pantothenic acid and its esters, panthenol, tetrahexydecyl ascorbate, ascorbyl glucosamine, magnesium ascorbyl phosphate, calcium ascorbate, L-ascorbic acid, folic acid and its derivatives, choline, camitine and the like. Astaxanthin may be used as a strong anti-oxidant. Any suitable vitamin or vitamin derivative can be used. It is preferred that at least some of the selected antioxidants, extracts and, or vitamins are stored and released in extended release delivery systems for increased lip enhancement effectiveness. Any suitable antioxidant, combinations of antioxidants, esters of antioxidants or derivatives thereof may be used as well.

Preferred embodiments having vitamin C would preferably also include the amino acids proline and lysine. Vitamin C is used up as it combines with the amino acids lysine and proline to form procollagen, which is then used to manufacture collagen in the body. Preferably, extended release delivery systems are arranged to replenish the supplies of useful vitamin C as well as lysine and proline in a predetermined manner to compensate for reduction in such compounds resulting from conversion to collagen synthesis, environmental degradation, or being flushed away from the vast network of blood vessels in the lips. It is preferred that concentrations of vitamin C, lysine and proline are sufficient to increase collagen synthesis in the tissues of the lip. Preferably, such increase in collagen synthesis is sufficient to create an increase in the volume of the lips.

Other amino acids may be included as well to further collagen synthesis, tissue growth and lip volume enhancement. It is preferred that amino acids are supplied within the composition in an amount effective to help stimulate and, or support collagen synthesis. Methionine is an essential amino acid that can be included since it can help supply sulfur for supporting healthy tissue formation. Threonine is an essential amino acid that can be included since it is important to the formation of collagen and elastin. Arginine is an non-essential amino acid that can be included since it promotes healing, tissue growth, and causes the release of growth hormones. Glycine is an non-essential amino acid that can be included since it helps trigger the release of oxygen to the energy required for new cell production and helps in hormone generation. Taurine is a non-essential amino acid that is preferably included since helps stabilize the excitability of membranes, helps control many biological changes that occur during aging, and help to expel free radical wastes. Cystine is a non-essential amino acid that is preferably included since it can function as an antioxidant to counter free radicals, protects against environmental pollution and toxins, can slow the aging process, and can aid in new skin formation (skin is 10-14% cystine). Other amino acids that can be used may include alanine, asparagine, aspartic acid, glutamine, glutamic acid, histadine, isoleucine, leucine, phenylalanine, proline, serine, trytophan, tyrosine, valine, oat amino acids as well as any other suitable sources of amino acids, hydrolyzed amino acids, free form amino acids, other suitable forms of amino acids or derivatives of amino acids. It is preferred that selected amino acid compounds are stored and released in extended release delivery systems for increased lip enhancement effectiveness.

It is also preferred that minerals are included in the composition that can provide support for new tissue formation and collagen production. One preferred mineral is copper, particularly organic forms of copper such as obtained by binding copper to peptides or small fragments of proteins so that the copper can be safely applied to the lips. The concentrations of copper peptides are preferably sufficient to increase collagen synthesis. Copper peptides can be used to increase collagen synthesis and to help smooth wrinkles and creases within the skin surrounding the lips as well as the skin of the lips. Other possible minerals may include iron, phosphorus, iodine, magnesium, selenium, copper, calcium, potassium and colloidal minerals. Any other nutritive or beneficial mineral or mineral compositions can also be included in the lip enhancing composition. It is preferred that selected mineral compounds are stored and released in extended release delivery systems for increased lip enhancement effectiveness.

It is also preferred that growth factors, hormones, hormone-like substances, and hormone stimulating substances are included. These compounds are preferably used in the lip enhancing composition to create external signals to the cell tissues for triggering increased collagen synthesis. Such substances can be used to provide signaling molecules for activating collagen formation for increased effectiveness and rate of lip volume augmentation. Any such compound may be used. Examples of such compounds may include growth factors, growth factor-1 (IGF-1) and/or growth factor-2 (IGF-2), somatomedin C, somatotropin (HGH), IGF-1 derived from the animal genus Cervus, IGF-1 derived from extract of Cervi Parvum Cornuany, any suitable ingredient or secretagogue capable of increasing the synthesis and release of IGF-1, any suitable ingredient or secretagogue capable of increasing the synthesis and release of human grown hormone (hGF), symbiotropin, anterior pituitary peptides, sequenced glycoamino acid complex, pharmaceutical saccharides, plant derived sources of L-Dopa, and botanical regulators of insulin and IGF-1. Other ingredients can include tretinoin, aminophylline, topical estrogens, phytoestrogen containing herbs such as black cohosh (isoflavones), soy isoflavones, fennel (anethole), wild yam (diosgenin), fenugreek (diosgenin), damiana, dong quai, saw palmetto and chaste tree berry. Other ingredients may include adrenaline, epinephrine, norepinephrine or other similar compounds. It is preferred that selected growth factors, hormones, hormone-like substances and hormone stimulating substances are stored and released in extended release delivery systems for increased lip enhancement effectiveness.

In alternate embodiments used for reducing fine lines and wrinkles in areas of the skin other than the lips, concentrations of ingredients used to stimulate the formation of collagen, hyaluronic acid, elastin and, or glucosamnoglycans would preferable be sufficient to stimulate the formation of collagen, hyaluronic acid, elastin and, or glucosamnoglycans within the skin and surrounding tissues in an amount effective to create a reduction in the appearance of such fine lines and wrinkles. For example, such ingredients would preferably stimulate the synthesis of collagen in the tissues beneath fine lines and wrinkles in an amount effective to smooth out such fine lines and wrinkles. Preferably, such ingredients would be sufficient to create an increase in tissue volume beneath fine lines and wrinkles in an amount effective to reduce lift up such fine lines and wrinkles to reduce their appearance. Preferably, such collagen synthesis would occur in an amount sufficient to provide an effective and safe alternative to surgical procedures and injections. It is also preferred that the combination of such collagen formation stimulating ingredients and blood circulation stimulating ingredients are sufficient to generate new tissue beneath such fine lines and wrinkles due to both collagen stimulation and new blood vessel formation from angiogenesis in an amount effective to reduce the appearance of wrinkles and fine lines over repeated applications.

Other ingredients to the compositions may also include acids, beta hydroxy acids (BHA) and alpha-hydroxy acids (AHA). Lactic acid, for example, is an alpha-hydroxy axid which has one of the smallest molecule sizes for better penetration into the skin. It may be used to create irritation and increased blood flow, or it may be used to not penetrate into the irritation zone of the skin so that it does not cause irritation and redness. Lactic acid may be used to help smooth the signs of aging, improve skin's texture and softness, reduce the appearance of fine lines and wrinkles, fade surface discoloration, reduce oiliness, unclog pores, and clear blemishes. Alpha-hydroxy acids or exfoliating ingredients may be used to exfoliate old dry cells so that the composition may penetrate underlying cells with greater efficiency.

Preferred embodiments would also include ingredients for supplying moisture and hydration to the lips. Increased moisture and hydration to the lips can significantly increase lip volume, especially when blood flow stimulation and collagen stimulation are being provided by the composition. Sodium hyaluronate and hyaluronic acid can be used to hydrate the skin and tissues of the lips. Because hyaluronic acid can hold up to approximately 1000 times its own weight in moisture, it can be used to supply moisture to the lip tissues. Amino acids, fucogel, and glycoaminoglycan can be used to draw and lock water into tissues for increased hydration. Pectins may also be used to retain moisture. Omega 3, 6 and 9 fatty acids may be used. Chamomile extract can help sooth and hydrate tissues. Any suitable oils, moisturizers and hydration stimulating ingredients may be used. Tocopheryl acetate (vitamine E) can be used to keep skin oils fresh. Any suitable forms of tocopherols (gamma, delta, beta, and d-alpha tocopherol, including d-alpha tocopherol acid succinate for example) and, or any suitable forms of tocotrienols, (d-alpha, d-beta, d-gamma and d-delta tocotrienol for example), or any other suitable forms of vitamin E or their derivatives may be used as well. Algae peptides may be used to improve moisturization and hydration to the lip tissues. UVA/UVB sun screens may also be used to prevent tissue damage, dehydration, photo aging, and sunburn to the lips. UV protectants can include organic and non-organic ingredients such as sodium cocoyl aminoacids, octinoxate, oxybenzone, octisalate, avobenzone, homosalate, sarcosine, potassium aspartate, magnesium aspertate, titanium dioxide, Parisol 1789®, coffee extracts, coral extracts as well as any other suitable sunscreens or UV inhibitors.

Other moisturizing or hydrating ingredients can include emu oil, aquaphor, dipalmitoyl, plankton, helianthus annuus (sunflower) seed oil, carthamus tinctorius (safflower) seed oil, persea gratissima (avocado) oil, symphytum officinale extract, cucumis stivus (cucumber) fruit extracts, hedera helix (ivy) extract, coffea arabica (coffee) extract, viola tricolor extract, cydomethican, commiphora myrrha oil, mirth extract, cymbopogon schoenanthus oil, salvia officinalis (sage) oil, petrolatum, lanolin, hydrolyzed lupin extract, royal jelly, hydrolyzed rice extract, vegitable oil, mineral oil, citrus oils, japan wax, oatmeal, caprylic/capric triglycerides, methylparaben, propylparaben, aloe barbadensis leaf extracts, aloe vera juice, glycine (soybean), paraffin wax, microcrystalline wax, vorexin complex, peanut oil, monoi oils, flax seed oil, biotin, almond oil, almond extract, walnut oil, walnut extract, sunflower seed oil, macadmaian nut oil, moringa oleifera, lanolin, castor oil, babassu oil orbignya oleifera), apricot kernal oil, shea, shea butter, cocoa butter (theobroma cacao), beeswax (cera alba), lamolin wax, paraffin wax, other suitable waxes, hydrolized soy protein, hydrolyzed whey protein, egg protein, soy protein, distilled water, spring water, mineral water, salt water, purified water, kelp extracts, kelp protein, seaweed extracts, lecithin, camphor, xanthum gum, grape seed oil, jojoba oil, glycol, white lilly gel, coconut oils, gelatins, moisturizing colloids, caster oil, glycerin, mineral oil, mink oil, petrolatum, mortierella Oil, arginine hydrochloride, hydrolyzed keratin, sunflower (Helianthus Annuus), ylang ylang (Cananga Odorata) oil, rosemary (Rosmarinus Officinalis) Oil, Niaouli (Melaluca Viridiflora) Oil, Bergamot (Citrus aurantium bergamia) oil, cypress (Cupressus Sempervirents) oil, lemon (citrus medica limonum) oil, bitter orange (citrus aurantium amara) oil, lavender (lavandula hybrida) oil, rosa centifolia oil, corn oil, (sage) leaf oil, melaleuca leucadendryon cajaputi oil, plant extracts, seed extracts, or any suitable organic and inorganic compound capable of providing a moisturizing effect to the skin or surrounding tissues.

It is preferred that some of the selected moisturizing and hydrating agents are stored and released in extended release delivery systems for increased effectiveness.

Any suitable carrier may be used for the compositions. The topical compositions may contain one or more conventional ancillary ingredients used in conventional topical compositions. In lip volume enhancing embodiments, any suitable carrier may be used for topical application to the lips. In embodiments used for fine line and wrinkle reduction, any suitable carrier may be used for application to the skin. Suitable carriers may include gels, glosses, creams, liquid carriers, thin liquid carriers, thick liquid carriers, waxes, oils, oil based carriers, water based carriers, serums, suspensions, colloidal suspensions, lotions, or any other form of carrier suitable to a particular mode and location of application. For example, the composition may contain one or more humectants, surfactants, preservatives, thickeners, fragrances, flavors, colorants, vitamins, retinoids, emmolients, and/or other suitable ingredients. Suitable humectants may include glycerine, sorbitol, polyethylene glycol, collagen, and the like. Suitable surfactants may include sodium stearyl sulfate, diethonolamine cetyl sulfate, polyethylene glycol, isostearate, and the like. Suitable preservatives may include methyl paraben, propyl paraben, and the like. Suitable retinoids may include retinol, retinoic acid, retinolic acid, Retin-A®, retinyl palmitate, reinyl propionate, retinyl acetate, isotrentonin, and the like. Suitable thickeners may include xanthan gum, locust bean gum, guar gum, hydroxyproyl cellulose, hydroxyethyl cellulose, carbopol, gum acacia, vee-gum, and magnesium aluminum silicate. Suitable emollients may include silicone oils (such as cyclomehticone/dimethicone copolyol, dimethicone and cyclotetramethicone), mineral oil, coca butter, fatty acid esters, beeswax and lanolin.

Other ingredients can include dimethicone, propylene glycol, propylene glycol stearate SE, glyceryl, polyglyceryl methacrylate, cetyl alcohol, triethanolamine, glyceryl stearate SE, octyl palmitate, tribehenin, sorbitan isostearate, potassium sorbate, cetyl palmitate, tristearin, quaternium-18 hectorite, bentonite, styearyl heptanoate, butyl stearate, palmitate, bisabolol, bis-diglyceryl polyacyladipate-2, polybutene, polysorbate-60, mica, ozokerite, phenyl trimethicone, diazolidinyl urea, imidazolidinyl urea, polymethacrylate, glycereth 26, biosaccharide gum, cetyl octanoate, sodium hyaluronate, polysorbate 20, peg-8/SMDI copolymer, palmitoyl-oligopeptide, disodium EDTA, trisodium EDTA, sodium ascorbyl phosphate, phenoxyethanol, suitable salts, benzyl nicotinate, pryidoxine, DL phenylannine, hexanicotinate, PVP/headecen copolymer, isopropyl palmitate, ceresin, PEG-100, stearate, phenylbenzimidazole, methyl gluceth 20, SD Alchohol 40-B, octyl palmitate, glycolic acid, polysorbate 80, ceteth-56, polyoxamer 401, octyl palmitate, silica, mica, fiber, phenoxyethanol, glyceryl polymethacrylate, stearic acid, citric acid, sodium lactate, cyclomethicone, cetyl octanoate, butylene glycol, squalane, ethylparben, phenoxyethanol, stearyl glycyrrhetinate, sodium glutamate, witch hazel and other similar ingredients.

Any suitable flavour can be used. Examples can include mint, spearmint, carmel, chocolate, coconut, lemon, lime, cinnamon, vanilla, cherry, raspberry, strawberry, other berry flavors, tangerine, citrus flavors, any suitable fruit flavors, red or black licorice flavor or extract, and menthol. Sweetness can be varied according to desire with ingredients such as honey, corn syrup, fructose, sucrose, glucose, glycosides, stevia extracts, stevia leaf extracts, steviocides, sodium saccharine, aspartame, Splenda®, herbal sweeteners, xylitol, sorbital, and gymnema sylvestris (blocks taste of sugar). Any suitable fragrance may be used such as fruit, mint, spice extracts or floral fragrances. Other ingredients can include extracts of grape fruit, citrus fruit, pear, apple seed, apple, onion, basil, papaya, kava root, tree bark, tree sap, calendlila officinalis, carrot, ginger, wheat grass, garlic, soy, kiwi, strawberry, pineapple, peach, banana, cantelope, squash, raspberry, blueberry, yeast extracts, St. John's wort, extracts of tree shoots as well as any other botanical or animal extract.

Below are various examples of some preferred embodiments of compositions.

Example Composition #1

Effective amounts of:
capsicum (preferably with liposomes or other extended release delivery system),
cinnamon (preferably with liposomes or other extended release delivery system),
caffeine,
guarana,
mimosin,
sepitonic,
gatuline,
hyaluronic acid and/or sodium hyaluronate (preferably with liposomes or other extended release delivery system),
palmitoyl oligopeptide (preferably with liposomes or other extended release delivery system),
allantoin,
niacin,
methyl-sulfonyl-methane (MSM) (preferably with liposomes or other extended release delivery system or suspended in a suitable carrier),
vitamin C (preferably with liposomes or other extended release delivery system),
magnesium ascorbyl phosphate,
ascorbyl methylsilanol pectinate,
vitamine E,
amino acids proline and lysine (preferably with liposomes or other extended release delivery system),
other amino acids: methionine, threonine, arginine, glycine, taurine, and cystiene, copper peptides (preferably with liposomes or other extended release delivery system),
tretinoin,
chamomile,
tocopheryl acetate (vitamine E),
a suitable UVAIUVB sun screen, and
a suitable carrier.
   Example Composition #2

Effective amounts of:
capsicum (preferably with liposomes or other suitable extended release delivery system),
cinnamon (preferably with liposomes or other suitable extended release delivery system),
caffeine,
hyaluronic acid and/or sodium hyaluronate (preferably with liposomes or other suitable extended release delivery system),
palmitoyl oligopeptide
allantoin,
niacin,
methyl-sulfonyl-methane (MSM)
vitamin C (preferably with liposomes or other suitable extended release delivery system),
magnesium ascorbyl phosphate,
ascorbyl methylsilanol pectinate,
amino acids proline and lysine,
copper peptides,
tretinoin,
tocopheryl acetate (vitamine E), and
a suitable carrier.
   Example Composition #3

Effective amounts of:
capsicum (preferably with liposomes or other suitable extended release delivery system),
hyaluronic acid and/or sodium hyaluronate (preferably with liposomes or other suitable extended release delivery system),
methyl-sulfonyl-methane (MSM) (preferably with liposomes or other suitable extended release delivery system),
vitamin C (preferably with liposomes or other suitable extended release delivery system),
amino acids proline and lysine (preferably with liposomes or other suitable extended release delivery system),
copper peptides (preferably with liposomes or other suitable extended release delivery system),
tocopheryl acetate (vitamine E), and
a suitable carrier.
   Example Composition #4

Effective amounts of:
Capsicum,
hyaluronic acid and/or sodium hyaluronate,
methyl-sulfonyl-methane (MSM),
vitamin C,
amino acids proline and lysine,
copper peptides, and
a suitable carrier.
   Example Composition #5

Effective amounts of:
capsicum and/or cinnamon extract,
hyaluronic acid and/or sodium hyaluronate,
methyl-sulfonyl-methane (MSM),
vitamin C (preferably with a suitable environmental protection),
amino acids proline and lysine, and
a suitable carrier.
   Example Composition #6

Effective amounts of:
capsicum and, or cinnamon extract,
hyaluronic acid and/or sodium hyaluronate,
vitamin C,
amino acids proline and lysine (preferably with liposomes or other suitable extended release delivery system, or in suspension within a suitable carrier) and
a suitable carrier.
   Example Composition #7

Effective amounts of:
capsicum and, or cinnamon extract,
hyaluronic acid and/or sodium hyaluronate,
vitamin C,
amino acids proline and lysine,
vitamin E,
N-acetylcysteine,
a suitable form of selenium (such as glutathione peroxidase for an example),
glutathione,
vitamin E,
alpha-lipoic acid, and
and a suitable carrier.
   Example Composition #8

Effective amounts of:
capsicum and, or cinnamon extract (preferably with liposomes or other suitable extended release delivery system)
and a suitable carrier.
   Example Composition #9

Effective amounts of:
capsicum and, or cinnamon extract,
a suitable form of hyaluronic acid and/or sodium hyaluronate,
vitamin C,
amino acids proline and lysine,
and a suitable carrier.
   Example Composition #10

Effective amounts of:
capsicum and, or cinnamon extract,
vitamin C,
amino acids proline and lysine,
and a suitable carrier.
   Example Composition #11

Effective amounts of:
capsicum and, or cinnamon extract (preferably with liposomes or other suitable extended release delivery system),
a suitable form of hyaluronic acid,
and a suitable carrier.
   Example Composition #12

Effective amounts of:
capsicum,
methyl-sulfonyl-methane (MSM),
amino acids proline and lysine,
vitamin C (preferably with suitable environmental protection),
and a suitable carrier.
   Example Composition #13

Effective amounts of:
capsicum and, or cinnamon,
methyl-sulfonyl-methane (MSM) (preferably with liposomes or other suitable extended release delivery system),
amino acids proline and lysine,
vitamin C (preferably with suitable environmental protection),
and a suitable carrier.
   Example Composition #14

Effective amounts of:
capsicum and, or cinnamon,
methyl-sulfonyl-methane (MSM) (preferably with liposomes or other suitable extended release delivery system),
amino acids proline and lysine,
vitamin C,
suitable bioflavinoids,
and a suitable carrier.
   Example Composition #15

Effective amounts of:
capsicum and, or cinnamon,
methyl-sulfonyl-methane (MSM)
amino acids proline and lysine,
vitamin C (preferably with suitable environmental protection),
caffeine,
ginseng,
copper peptides,
suitable tocopherols and, or tocotrienols,
and a suitable carrier.
   Example Composition #16

Effective amounts of:
Growth factor-1 (IGF-1) also known as somatomedin C (preferably with any suitable type of extended release delivery system, such as liposomes as an example) amino acids proline and lysine,
vitamin C (preferably with suitable environmental protection),
and a suitable carrier.
   Example Composition #17

Effective amounts of:
capsicum and, or cinnamon,
Somatomedin C growth factor-1 (IGF-1) (preferably with any suitable type of extended release delivery system, such as liposomes as an example) amino acids proline and lysine,
vitamin C (preferably with suitable environmental protection such as bioflavinoids and/or an extended release delivery system, such as liposomes for example),
and a suitable carrier.
   Example Composition #18

Effective amounts of:
capsicum and, or cinnamon,
methyl-sulfonyl-methane (MSM)
Somatomedin C growth factor-1 (IGF-1) (preferably with any suitable type of extended release delivery system, such as liposomes as an example) amino acids proline and lysine,
vitamin C (preferably with suitable environmental protection such as bioflavinoids and/or an extended release delivery system, such as liposomes for example),
and a suitable carrier.
   Example Composition #19

Effective amounts of:
capsicum and, or cinnamon,
methyl-sulfonyl-methane (MSM)
Somatomedin C growth factor-1 (IGF-1) (preferably with any suitable type of extended release delivery system, such as liposomes as an example) caffeine,
hyaluronic acid and/or sodium hyaluronate (preferably with liposomes or other suitable extended release delivery system),
palmitoyl oligopeptide
allantoin,
niacin,
vitamin C (preferably with liposomes or other suitable extended release delivery system),
magnesium ascorbyl phosphate,
ascorbyl methylsilanol pectinate,
amino acids proline and lysine,
copper peptides,
tretinoin,
astaxanthin,
tocopheryl acetate (vitamine E), and
a suitable carrier.
   Example Composition #19

Effective amounts of:
capsicum and, or cinnamon,
methyl-sulfonyl-methane (MSM)
Somatomedin C growth factor-1 (IGF-1) (preferably with any suitable type of extended release delivery system, such as liposomes as an example) caffeine,
hyaluronic acid and/or sodium hyaluronate (preferably with liposomes or other suitable extended release delivery system),
palmitoyl oligopeptide
allantoin,
niacin,
vitamin C (preferably with liposomes or other suitable extended release delivery system),
amino acids proline and lysine,
copper peptides,
tretinoin,
astaxanthin,
tocopheryl acetate (vitamine E), and
a suitable carrier.
   Example Composition #20

Effective amounts of:
Somatomedin C growth factor-1 (IGF-1) (preferably with any suitable type of extended release delivery system, such as liposomes as an example) vitamin C (preferably with liposomes or other suitable extended release delivery system),
amino acids proline and lysine, and
a suitable carrier.
   Example Composition #21

Effective amounts of:
capsicum and, or cinnamon,
Somatomedin C growth factor-1 (IGF-1) (preferably with any suitable type of extended release delivery system, such as liposomes as an example),
hyaluronic acid and/or sodium hyaluronate, and
a suitable carrier.
   Example Composition #22

Effective amounts of stevia extracts, stevia leaf extracts, or steviocides can be used within a suitable topical carrier to provide increased blood circulation to the skin, to stimulate collagen synthesis, to reduce the appearance of wrinkles and fine lines in the skin, or to provide improved firmness, elasticity or moisturize retention to the skin in moisturizing compounds.

Example Composition #23

Effective amounts of:
capsicum and, or cinnamon,
stevia extracts, stevia leaf extracts, or steviocides
Somatomedin C growth factor-1 (IGF-1) (preferably with any suitable type of extended release delivery system, such as liposomes as an example),
hyaluronic acid and/or sodium hyaluronate, and
a suitable carrier.
   Example Composition #24

Effective amounts of:
capsicum and, or cinnamon,
stevia extracts, stevia leaf extracts, or steviocides vitamin C (preferably with liposomes or other suitable extended release delivery system),
amino acids proline and lysine,
hyaluronic acid and/or sodium hyaluronate, and
a suitable carrier.
   Example Composition #25

Effective amounts of:
stevia extracts, stevia leaf extracts, or steviocides vitamin C (preferably with liposomes or other suitable extended release delivery system), amino acids proline and lysine, and a suitable carrier.

When the above examples or other alternate embodiments are used for increasing lip volume, it is preferred that the concentrations of the ingredients are sufficient to create an increase in the volume of the lips. When the above examples or other alternate embodiments are used for reducing fine lines and wrinkles to other areas of the skin, it is preferred that the concentrations of the ingredients are sufficient to reduce the appearance of fine lines and wrinkles in the skin. When the above examples or other alternate embodiments are used to reduce fine lines and wrinkles, increased concentrations of ingredients can be used for focused applications in which a predetermined composition is applied specifically to fine lines and wrinkles for localized stimulation of tissue formation below and around the fine lines and wrinkles. When the above examples or other alternate embodiments are used as a general skin moisturizer, concentrations are preferably sufficient to create increased moisture, elasticity and vitality to the skin. These and other compounds may be used to enhance the skin in an amount sufficient to either increase blood flow, increase tissue formation, increase tissue volume, increase skin moisture, increase skin elasticity, increase skin tone, increase skin firmness, increase skin vitality, increase collagen formation, increase elastin information, increase lip size, reduce the appearance of fine lines, reduce the appearance of wrinkles, or reduce the depth of wrinkles.

### SUMMARY, RAMIFICATIONS AND SCOPE

Accordingly, the reader will see that the compositions and methods of the present invention can be used to provide improved lip volume enhancement and improved fine line and wrinkle reduction to the skin on and around the lips as well as to areas of skin not on or near the lips. The reader will also see that the methods of present invention can be used to significantly increased blood circulation and angiogenesis (new blood vessel generation) as well as increased collagen synthesis over extended periods of time for increased effectiveness. Furthermore, the methods and compositions of the present invention offer additional advantages in that they:
(a) provide topical lip volume enhancement methods and compositions that offer increased effectiveness;
(b) provide methods for extended release and automatic replenishment of lip volume enhancing compounds over extended durations so as to significantly reduce the need for reapplication;
(c) provide topical lip volume enhancement methods and compositions that provide significantly long durations of increased lip volume;
(d) provide methods for increasing the duration of lip tissue swelling in an amount effective to permit the lip tissues to expand and adjust to the increased volume so as to reduce the amount of elastic recovery after swelling and to permit lip tissues to experience increased net gain in volume over time;
(e) provide methods for storing and releasing lip volume enhancing compounds and lip tissue nourishing compounds over extended periods with extended release delivery systems so as to substantially overcome the low storage capacity of compounds on and within the thin skin layers of the lips;
(f) provide methods for storing and releasing lip volume enhancing compounds and lip tissue nourishing compounds over extended periods with extended release delivery systems so as to substantially permit the automatic extended replenishment of lip volume enhancing compounds and lip tissue nourishing compounds to the blood vessels and tissues beneath the thin skin of the lip as the vast network of blood vessels beneath this thin skin of the lips begins to absorb and carry such lip volume enhancing compounds and lip tissue nourishing compounds away from the lip tissues;
(g) provide methods improving the effectiveness of topically applied blood circulation enhancing compounds that normally taper off in effectiveness after a relatively short period of time, in which additional amounts of such compounds are stored within extended release delivery systems for automatic re-application on a time release basis substantially coinciding with the time that the original unstored compounds begin to taper off in effectiveness, or coinciding with the time that previously released stored circulation enhancing compounds begin to taper off in effectiveness, thereby enabling such short period blood circulation enhancing compounds to be effective for significantly increased periods capable of creating longer durations of increased blood flow, lip volume enhancement and lip tissue reorganization;
(h) provide methods for extended release delivery of blood flow enhancing compounds for increasing the duration of increased circulation and swelling to the lip tissues in an amount effective to increase new blood vessel formation within the lip tissues;
(i) provide methods for extended release delivery of topical collagen and hyaloronic acid production formation stimulating compounds in an amount effective to increase the formation of new collagen for increased tissue formation within the lips;
(j) provide methods for extended release delivery of blood circulation enhancing compounds and topical collagen and hyaloronic acid production stimulation compounds to the lip in an amount effective to increase the formation of new collagen and hyaloronic acid within the lips during the time that the extended release of blood circulation enhancing compounds are creating an extended increase lip volume, thereby permitting such collagen and hyaloronic acid to begin forming while the lip tissues are in an expanded condition for greater lip enhancement effectiveness;
(k) provide methods for extended release delivery of blood circulation enhancing compounds and collagen and hyaloronic acid formation stimulating compounds within a carrier containing hydration enhancing compounds for delivering simultaneous hydration to the lip tissues blood circulation stimulation and collagen formation stimulation while providing hydration enhancing compounds maximizing lip hydration;
(l) provide methods and compounds that store and release capsicum with at least one extended release delivery system disposed within a topical carrier for application to the lips and arranged to provided an extended release of such capsicum in an amount and duration effective to significantly increase lip size and volume due to swelling and increase blood flow to the lip tissues;
(m) provide methods and compounds that store and release methyl-sulfonyl-methane with at least one extended release delivery system disposed within a topical carrier for application to the lips and arranged to provided an extended release of such methyl-sulfonyl-methane in an amount and duration effective to increase elasticity of the lip tissues;
(n) provide methods and compounds that store and release of methyl-sulfonyl-methane with at least one extended release delivery system disposed within a topical carrier for application to the lips and arranged to provided an extended release of such methyl-sulfonyl-methane in an amount and duration effective to stimulate collagen and hyaloronic acid formation within the lip tissues;
(o) provide growth factors and, or growth hormones for increasing collagen synthesis and new tissue formation;
(p) provide methods and topical compositions which offer improved ability to reduce the appearance of aging in the skin;
(q) provide methods and topical compositions which offer improved reduction of wrinkles and fine lines in the skin; and
(r) to provide methods and topical compositions capable of improving the formation of collagen and hyaluronic acid within and around the tissues of the skin.

While the above description provides examples of some of the preferred embodiments and preferred ingredients, any of the ingredients or category of ingredients provided in this description may be used with each other or with any other ingredients in any combination, concentration, and amount. In addition, any type of suitable carrier may be used as a vehicle for such ingredients. While it is preferred that an extended release delivery system is used for at least one of the ingredients in a given composition, alternate coinpositions may avoid the use of any extended release delivery system.

In embodiments in which extended release delivery systems are used, the extended release of blood circulation ingredients and collagen formation stimulating ingredients may be arranged to create a synergistic effect by maintaining increased blood flow and collagen synthesis over extended periods so that new tissue may be formed during periods of extended tissue swelling created by the increased blood flow. This can allow an increased volume of tissue to form during a period of swelling and such increased volume of tissue can cause the tissues to achieve an increased long term volume when the period of swelling ends, similar to putting one's foot in the way of a closing door so that the door does not close all the way. Over repeated applications, the tissue volume can continue to increase and reach new increased equilibrium volumes after the periods of prolonged swelling subside. This can cause lip volume enhancement to be improved. The increased blood flow can also provide increased nutrients to the region for increased tissue generation. In any of the embodiments, the use of liposomes can allow the delivery of collagen formation stimulating ingredients and/or blood circulation stimulating ingredients to take place directly to the intended cells and can occur over a longer period of time. This can allow liposomes and lip volume enhancing compositions to create an increased synergistic effect of increased lip volume enhancement. The period of time for the extended release of collagen formation stimulating ingredients in any embodiment can be arranged to be shorter, equal to or longer than the period of time for the extended release of blood flow circulation ingredients and, or the time period of other extended release ingredients. In alternate embodiments used for reducing fine lines and wrinkles, the increased blood flow can be sufficient to increase collagen formation, increased tissue volume below the fine lines and wrinkles.

Alternate embodiments may also include oral compositions to be taken orally for internal support of skin enhancement, either simultaneously with topical compositions, at different times as topical composition embodiments or alone without any use of topical composition embodiments. Other alternate embodiments can include improved compositions arranged for injection below the skin, either together with topical compositions and, or oral embodiments, or alone without the use of topical or oral embodiments. Still other embodiments can include compositions arranged for intravenous injections or delivery.

While the above description contains many specificities, these should not be construed as limitations on the scope of the invention, but rather as an exemplification of several preferred embodiments thereof. Many other variations are possible. For example: all of the described methods, embodiments, and variations can be combined with one another in any desired order, amount or configuration. Any of the ingredients may stand alone or in any combination with any other ingredient or ingredients.

In alternate embodiments, Effective amounts of the compound ingredients may be used to reduce the appearance of scars, blotches in skin color or texture, acne, skin maladies, skin diseases, or other skin problems. Alternate embodiments may also be used for reducing hair loss or stimulating hair growth. Alternate embodiments may be used in oral rinses or toothpastes to stimulate healthy gum and mouth tissues or to heal mouth tissues, sores or other oral problems. Alternate embodiments may also use the methods, compositions and ingredients to improve make up, lip stick, lip gloss, sun tan lotion, sun block lotion, night creams, day creams, hand lotions, skin cleansers, exfoliation compositions and other cosmetic products.

Accordingly, the scope of the invention should be determined not by the embodiments illustrated and discussed, but by the appended claims and their legal equivalents.

1. A topically administered composition for enhancing the skin of a human, comprising:
(a) a predetermined carrier suitable for topical application to the skin of a human arranged to store and deliver predetermined skin enhancing ingredients;
(b) an effective amount of vitamin C;
(c) an effective amount of a predetermined protective component arranged to protect said effective amount of vitamin C from experiencing oxidation damage prior to delivery of said effective amount of vitamin C to said skin,
(d) an effective amount of proline;
(e) an effective amount of lysine;
(f) an effective amount of a form of hyaluronic acid selected from the group consisting of hyaluronic acid, non-toxic salts related to hyaluranic acid, derivatives thereof and combinations thereof; and
(g) an effective amount of at least one predetermined blood circulation stimulating ingredient capable of increasing blood circulation within said skin.

2. The composition of claim 1 wherein said at least one predetermined blood circulation stimulating ingredient is selected from the group consisting of capsicum, capsaicin, cinnamon extract, cinnamon bark extract and caffeine.

3. The composition of claim 1 wherein said effective amount of at least one predetermined blood circulation stimulating ingredient is an effective amount of capsicum.

4. The composition of claim 3 wherein said effective amount of capsicum is stored and released in a predetermined extended release delivery system.

5. The composition of claim 4 wherein said extended release delivery system is a liposome.

6. The composition of claim 4 wherein said predetermined extended release delivery system is arranged to release said at least one portion of said composition over an extended release period of at least 30 minutes.

7. The composition of claim 6 wherein said extended release period is not less than 1 hour.

8. The composition of claim 1 wherein at least one portion of said composition is arranged to be stored and released with a predetermined extended release delivery system.

9. The composition of claim 1 wherein said predetermined protective component is selected from the group consisting of flavinoids, bioflavinoids, liposomes, predetermined extended release delivery systems, silicon, peroxidase, alpha-lipoic acid, glutathione, N-acetylcysteine, selenium, tocotrienols and their derivatives, tocopherols and their derivatives, and vitamin E.

10. The composition of claim further including an effective amount of copper peptides.

11. The composition of claim 1 wherein said composition is sufficiently concentrated to significantly reduces fine lines and wrinkles within said skin.

12. The composition of claim 1 wherein said composition arranged to be applied to the lips of said human and at least one portion of said composition is arranged to have a concentration effective to permit said composition to create an increase in the size of said lips.

13. A method for enhancing the size of the lips of a human, comprising:
(a) providing a predetermined carrier arranged for application to the lips of a human;
(b) providing said predetermined carrier with an effective amount of capsicum capable of creating visible swelling to said lips sufficient to enhance the size of said lips; and
(c) storing and releasing said capsicum within a predetermined extended release delivery system arranged to release and deliver said capsicum to said lips for an extended release period of at least 30 minutes after said application to said lips so as to significantly increase the duration of said swelling.

14. The method of claim 14 wherein a sufficient amount of at least one collagen formation stimulating ingredient is added to said carrier to increase the formation of collagen in said lips so as to create long term increase in the volume of said lips.

15. The method of claim 14 wherein said at least one collagen stimulating ingredient is selected from the group consisting of collagen, elastin, hyaluronic acid, collagen precursors, procollagens (such as procollagen type I for example), hydroxproline, palmitoyl oligopeptide, hyaluronic acid salts (such as sodium salts), a suitable form of hyaluronic acid, sodium hyaluronate, allantoin, niacin, vitamin C, alpha-lipoic acid, proline, lysine, and palmitoyl oligopeptide.

16. The method of claim 14 wherein said extended release period is not less than 45 minutes.

17. The method of claim 14 wherein said extended release period is not less than 1 hour.

18. The method of claim 14 wherein said extended release period is not less than 2 hours.

19. The method of claim 15 wherein said at least one collagen formation stimulating ingredient is stored within a separate extended release delivery system.

20. A topical composition for enhancing the size of the lips of a human, comprising:
(a) a predetermined carrier arranged for application to the lips of a human;
(b) an effective amount of capsicum to create visible swelling to said lips sufficient to enhance the size of said lips, said capsicum being stored and released in a predetermined extended release system arranged to release said capsicum over an extended release period of at least 30 minutes after said application;
(c) an effective amount of a suitable form of hyaluronic acid;
(d) an effective amount of vitamin C;
(e) an effective amount of proline;
(f) an effective amount of lysine; and
(g) an effective amount of a suitable form of vitamin E.

21. The topical composition of claim 20 wherein said suitable form of hyaluronic acid is stored and released in with a separate predetermined extended release system.

22. The topical composition of claim 20 wherein said vitamin C is stored and released with a liposome.

23. The topical composition of claim 20 wherein said extended release period is not less than 1 hour.

24. The topical composition of claim 20 further including an effective amount of palmitoyl oligopeptide.

25. The topical composition of claim 24 wherein said effective amount of palmitoyl oligopeptide is stored and released from a separate predetermined extended release system.
